(19) United States
(12) Patent Application Publication
(10) Pub. No.: US 2020/0178542 A9
(43) Pub. Date: Jun. 11, 2020

(54) CONTROL OF PESTS IN COTTON BY GINKGOLIDES AND BILOBALIDES

(71) Applicant: BASF SE, Ludwigshafen am Rhein (DE)

(72) Inventors: Birgit Gockel, Ludwigshafen (DE); Joachim Dickhaut, Heidelberg (DE); Daniel Saelinger, Ludwigshafen (DE)

(21) Appl. No.: 15/754,074

(22) PCT Filed: Aug. 8, 2016
(86) PCT No.: PCT/EP2016/068839

§ 371 Date: Feb. 21, 2018

(30) Foreign Application Priority Data
Aug. 25, 2015 (EP) 15182261.6

(51) Int. Cl. A01N 65/00; A01H 1/06; A01H 6/60

(52) U.S. Cl. CPC A01N 65/00; A01H 1/02; A01H 6/604; A01H 1/06

(57) ABSTRACT

A method for controlling pests of a cotton plant is disclosed. The method includes the step of contacting the cotton plant, parts of it, or its propagation material with components of the ginkgo tree selected from bilobalide, ginkgolide A, ginkgolide B, ginkgolide C, ginkgolide J, ginkgolide M, and mixtures thereof. The use of components of the ginkgo tree for controlling pests in a cotton plant, parts of it, or its propagation material, is also disclosed. A pesticide including one or more components of the ginkgo tree selected from bilobalide, ginkgolide A, ginkgolide B, ginkgolide C, ginkgolide J, and ginkgolide M is further disclosed.

The invention relates to a method for controlling pests of a cotton plant (Gossypium), comprising the step of contacting the cotton plant, parts of it, its propagation material, the pests, their food supply, habitat or breeding grounds with components of the ginkgo tree selected from bilobalide, ginkgolide A, ginkgolide B, ginkgolide C, ginkgolide J, ginkgolide M, and mixtures thereof. The invention also relates to the use of one or more components of the ginkgo tree selected from bilobalide, ginkgolide A, ginkgolide B, ginkgolide C, ginkgolide J, ginkgolide M, and mixtures thereof, for controlling pests in cotton plants.

The damages caused by insects on cotton harvests, e.g. insects from the superfamilies of Pentatomidae—comprehensively referred to as stink bugs—and/or Thripidae, represents a huge economical risk to the farmer. The family of Pentatomidae comprises Acrosternum spp., Euschistus spp., Halyomorpha spp., Nezara spp. and/or Megacopta spp., which are all of extraordinary relevance on cotton plants. Typical species found in cotton are Euschistus servus (brown stink bug), Euschistus heros (Neotropical brown stink bug), Halyomorpha halys (brown marmorated stink bug), Nezara viridula (southern green stink bug), Megacopta cribraria (globular stink bug), and Piezodorus guildini. Thripidae are also of exceptional relevance to cotton plants. Infection, e.g. by Frankliniella spp. occurs both on seeds, seedlings and young plants. Frankliniella occidentalis, and Frankliniella williamsi represent some of the most aggressive pests on cotton.

Insecticides commonly used to control stinkbugs include pyrethroids, neonicotinoids and organophosphates. However, there are increasing problems with insecticide resistance, particularly in brown stink bug populations and especially to neonicotinoids and pyrethroids, in particular to pyrethroids. Euschistus heros can also be difficult to manage using organophosphates or endosulfan (Sosa-Gomez et al., 2009). There is therefore a need for effective methods of controlling stinkbugs in cotton, especially for overcoming resistance problems. Furthermore, the stricter regulatory requirements on pesticides causes a need for pesticides with lower toxicities, and enhanced environmental safety, e.g. lower pesticide residues, higher target selectivity, reduced impact on non-pest animals (e.g. bees), improved aquatic safety, and reduced dose rates. On top, there is a need for pesticides that are able to control pests that have become resistant to insecticidal traits of genetically modified cotton plants.

Ginkgolides and bilobalides are natural products that are produced by the Ginkgo tree. An acaricidal activity of ginkgolide C is disclosed in CN 102379296 (A). WO 2005/025587 discloses an insecticidal activity of mixtures of ginkgolide A, B and bilobalide against two spotted spider mites and green peach aphid. WO 2004/034853 discloses insecticidal mixtures of bilobalide, ginkgolide A, B or C and unspecified derivatives thereof with organophosphates and pyrethroids against rice pests. WO2015/128338 discloses uses and methods of application of ginkgolides and bilobalides against Pentatomidae and/or Thripidae on Faboidae.

In view of the importance of cotton in agriculture, it was an objective to supply uses and methods of application for pest control in cotton, especially of pests selected from Pentatomidae, and Thripidae, and in particular of pests that have become resistant to another insecticide, or an insecticidal trait of a genetically modified cotton plant.

It has now surprisingly been found that ginkgolides and/or bilobalides selected from bilobalide, ginkgolide A, ginkgolide B, ginkgolide C, ginkgolide J, ginkgolide M, and mixtures thereof, are exceptionally suitable to address the above problems and needs. These compounds therefore represent an important solution for controlling pests of cotton, especially pests from the families of Pentatomidae and/or Thripidae, and thereby safeguarding plants, and propagation material from the infestation by such pests, particularly where the pests have become resistant to other insecticides, or insecticidal traits of genetically modified plants. In particular, it has surprisingly been found that the application of these compounds on cotton plants having insecticidal traits is highly effective for combating pests on these cotton plant, such as pests that have become resistant to the insecticidal traits.

Bilobalide and the ginkgolides are known components of the ginkgo tree having the following structures:
a) Bilobalide:

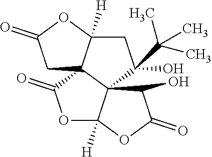

Bilobalide is the common name for (3aS,5aR,8aS,9R,10aR)-9-tert-butyl-8,9-dihydroxydihydro-9H-furo[2,3-b]furo[3′,2′; 2,3]cyclopenta[1,2-c]furan-2,4,7(3H,8H)-trione (CAS 33570-04-6).
b) Ginkgolides:

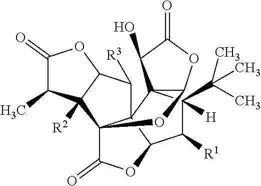

TABLE A
Definitions of substituents for Ginkgolides

| Ginkgolide | R¹ | R² | R³ | CAS |
|---|---|---|---|---|
| Ginkgolide A | H | OH | H | 15291-75-5 |
| Ginkgolide B | H | OH | OH | 15291-77-7 |
| Ginkgolide C | OH | OH | OH | 15291-76-6 |
| Ginkgolide J | OH | OH | H | 15291-79-9 |
| Ginkgolide M | OH | H | OH | 15291-78-8 |

The compounds of Table A can be used in pure form, as mixtures or in the form of extracts of ginkgo leaves, which may be enriched with the above compounds to a certain degree. The components of the ginkgo tree in Table A are also referred to as “components of the ginkgo tree”. Accordingly, when referring to the components of the ginkgo tree, the term relates to at least one of the compounds of Table A, or a mixture thereof.

The compounds are commercially available, or can be obtained, preferably from ginkgo leaves by methods known in the art and described e.g. in U.S. Pat. No. 5,700,468, EP-A 360 556, EP-A 0 431 535 and JP-A 09-110713. Further, the compounds bilobalide (in enantiopure form), ginkgolide A (in its racemic form) and ginkgolide B (in its racemic form) can be obtained by chemical synthesis, as disclosed e.g. in Tetrahedron Letters (1988), 29(28), 3423-6, Tetrahedron Letters (1988), 29(26), 3205-6 and Journal of the American Chemical Society (2000), 122(35), 8453-8463, respectively.

Within this application, combinations of embodiments and/or preferences with other embodiments and/or preferences are within the scope of the invention, regardless of the level of preference of the respective features.

The uses and methods disclosed herein relate to the application of bilobalide, ginkgolide A, ginkgolide B, ginkgolide C, ginkgolide J, ginkgolide M, or mixtures thereof. In one embodiment, the disclosed uses and methods relate to bilobalide and/or ginkgolide A, or mixtures thereof. In another embodiment, the disclosed uses and methods relate to bilobalide. In another embodiment, the disclosed uses and methods relate to ginkgolide A, ginkgolide B, ginkgolide C, ginkgolide J, ginkgolide M, or mixtures thereof. In another embodiment, the disclosed uses and methods relate to ginkgolide A. In another embodiment, the disclosed uses and methods relate to ginkgolide B. In another embodiment, the disclosed uses and methods relate to ginkgolide C. In another embodiment, the disclosed uses and methods relate to ginkgolide J. In another embodiment, the disclosed uses and methods relate to ginkgolide M.

The methods and uses of the invention are for controlling pests of a cotton plant, preferably by Pentatomidae and/or Thripidae. In one embodiment, the methods and uses of the present invention are applied for controlling pests from the family of Pentatomidae. In another embodiment, the methods and uses of the present invention are applied for controlling pests from the family of Thripidae.

In one embodiment, the methods and uses of the present invention relate to a cotton plant, which has been modified by conventional breeding, i.e. a plant, which has not been modified by mutagenesis or genetic engineering. In another embodiment, the methods and uses of the present invention relate to a genetically modified cotton plant. In another embodiment, the components of the ginkgo tree are applied for controlling pests that are resistant to one or more other insecticides, preferably pyrethroids, neonicotinoids and organophosphates, and more preferably pyrethroid insecticides. In another embodiment, the components of the ginkgo tree are applied for controlling pests that are resistant to an insecticidal trait of a genetically modified cotton plant.

The term “genetically modified” generally refers to mutagenesis or genetic engineering techniques. In one embodiment, the term refers to mutagenesis. In another embodiment, the term refers to genetic engineering. In yet another embodiment, the term refers to a combination of genetic engineering with conventional breeding. In yet another embodiment, the term refers to a combination of mutagenesis with conventional breeding.

The term “conventional breeding” refers to methods comprising crossing (i.e. utilizing meiosis) and/or selection, e.g. crossbreeding, mutagenesis (excluding induction by mutagens or directed mutagenesis), or recombination (such as sexual recombination, but excluding directed recombination by introduction of DNA fragments produced by methods of genetic engineering). Conventional breeding may include the application of selection markers, such as herbicide tolerance. Typical plants that are obtainable by conventional breeding are plants of the Clearfield™ product line, such as Clearfield™ cotton. Such plants obtainable by conventional breeding may carry a herbicide tolerance, preferably against Imazethapyr, or Imazapyr.

Usually, genetically modified plants as referred to in this application carry a gene of a different organism, which gene is also referred to as transgene. The skilled person is able to select suitable techniques to produce genetically modified plants, and to analyze plants on genetic modifications.

Cotton plants that are “resistant” against insects display a lower degree of infestation by at least one insect species compared to a cotton plant of the same variety. Such resistance may be achieved by conventional cultivation techniques, or by mutagenesis and/or genetic engineering. Pests that are “resistant” to a particular insecticide, or insecticidal trait of a cotton plant are less sensitive to that insecticide, or insecticidal trait of a cotton plant compared to the same pest species, in particular compared to the same subspecies. Accordingly, Pentatomidae that are “resistant” to a particular insecticide, or insecticidal trait of a cotton plant refers e.g. to strains of Pentatomidae that are less sensitive to that insecticide, or insecticidal trait of a cotton plant compared to the expected sensitivity of the same species of Pentatomidae. The expected sensitivity can be measured using e.g. a strain that has not previously been exposed to the insecticide, or the insecticidal trait of a genetically modified cotton plant.

In one embodiment, the components of the ginkgo tree are used to control and/or prevent infestation by Thripidae, preferably Frankliniella spp. (e.g. Frankliniella occidentalis, Frankliniella williamsi). In another embodiment, the components of the ginkgo tree are applied for controlling and/or preventing infestation by Acrosternum spp., Euschistus spp., Halyomorpha spp., Nezara spp., Megacopta spp., and Piezodorus spp. In yet another embodiment, the components of the ginkgo tree are applied for controlling and/or preventing infestation Euschistus spp., Halyomorpha spp., Megacopta spp., or Nezara spp. In yet another embodiment, the components of the ginkgo tree are applied for controlling and/or preventing infestation by Euschistus spp., Halyomorpha spp., or Megacopta spp. In yet another embodiment, the components of the ginkgo tree are applied for controlling and/or preventing infestation by Euschistus spp. In yet another embodiment, the components of the ginkgo tree are applied for controlling and/or preventing infestation by Halyomorpha spp. In yet another embodiment, the components of the ginkgo tree are applied for controlling and/or preventing infestation by Piezodorus spp. In yet another embodiment, the components of the ginkgo tree are applied for controlling and/or preventing infestation by Megacopta spp. In yet another embodiment, the components of the ginkgo tree are applied for controlling and/or preventing infestation by Nezara spp. In yet another embodiment, the components of the ginkgo tree are applied for controlling and/or preventing infestation by Acrosternum spp. In one embodiment, the components of the ginkgo tree are applied for controlling and/or preventing infestation by Acrosternum hilare, Euschistus servus, Euschistus heros, Halyomorpha halys, Megacopta cribraria, Nezara viridula, or Piezodorus viridula. In another embodiment, the components of the ginkgo tree are applied for controlling and/or preventing infestation by Euschistus heros, Euschistus servus, Halyomorpha halys, Megacopta cribraria, or Nezara viridula. In yet another embodiment, the components of the ginkgo tree are applied for controlling and/or preventing infestation by Euschistus servus. In yet another embodiment, the components of the ginkgo tree are applied for controlling and/or preventing infestation by Euschistus heros. In yet another embodiment, the components of the ginkgo tree are applied for controlling and/or preventing infestation by Halyomorpha halys. In yet another embodiment, the components of the ginkgo tree are applied for controlling and/or preventing infestation by Acrosternum hilare. In yet another embodiment, the components of the ginkgo tree are applied for controlling and/or preventing infestation by Megacopta cribraria. In yet another embodiment, the components of the ginkgo tree are applied for controlling and/or preventing infestation by Nezara viridula. In yet another embodiment, the components of the ginkgo tree are applied for controlling and/or preventing infestation by Piezodorus viridula. In another embodiment, the pests are animal pests such as arthropods, gastropods and nematodes including but not limited to: insects from the order of Lepidoptera, for example Achroia grisella, Acleris spp. such as A. fimbriana, A. gloverana, A. variana; Acrolepiopsis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia (=Thermesia) spp. such as A. gemmatalis; Apamea spp., Aproaerema modicella, Archips spp. such as A. argyrospila, A. fuscocupreanus, A. rosana, A. xyloseanus; Argyresthia conjugella, Argyroploce spp., Argyrotaenia spp. such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. murinana, C. podana; Cactoblastis cactorum, Cadra cautella, Calingo braziliensis, Calopas theivora, Capua reticulana, Carposina spp. such as C. niponensis, C. sasakii; Cephus spp., Chaetocnema aridula, Cheimatobia brumata, Chilo spp. such as C. Indicus, C. suppressalis, C. partellus; Choreutis pariana, Choristoneura spp. such as C. conflictana, C. fumiferana, C. longicellana, C. murinana, C. occidentalis, C. rosaceana; Chrysodeixis (=Pseudoplusia) spp. such as C. eriosoma, C. includens; Cirphis unipuncta, Clysia ambiguella, Cnaphalocerus spp., Cnaphalocrocis medinalis, Cnephasia spp., Cochylis hospes, Coleophora spp., Collas eurytheme, Conopomorpha spp., Conotrache/us spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabills, D. sibiricus; Desmia funeralis, Diaphania spp. such as D. nitidalis, D. hyalinata; Diatraea grandiosella, Diatraea saccharalis, Diphthera Festiva, Earias spp. such as E. insulana, E. vittella; Ecdytolopha aurantianu, Egira (=Xylomyges) curialis, Elasmopalpus lignosellus, Eldana saccharin, Endopiza viteana, Ennomos subsignaria, Eoreuma loftini, Ephestia spp. such as E. cautella, E. elutella, E. kuehniella; Epinotia aporema, Epiphyas postvittana, Erannis tiliaria, Erionota thrax, EtieIla spp., Eulia spp., Eupoecilla ambiguella, Euproctis chrysorrhoea, Euxoa spp., Evetria bouliana, Faronta albilinea, Feltia spp. such as F. subterranean; Galleria mellonella, Gracfilarla spp., Grapholita spp. such as G. funebrana, G. molesta, G. inopinata; Halysidota spp., Harrisina americana, Hedylepta spp., Helicoverpa spp. such as H. armigera (=Heliothls armigera), H. zea (Heliothls zea); Hellothis spp. such as H. assulta, H. subflexa, H. virescens; Hellula spp. such as H. undalis, H. rogatalis; Helocoverpa gelotopoeon, Hemileuca oliviae, Herpetogramma licarsisalis, Hibernia defoliaria, Hofmannophila pseudospretella, Homoeosoma electellum, Homona magnanima, Hypena scabra, Hyphantria cunea, Hyponomeuta padella, Hyponomeuta malinellus, Kakivoria fiavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocolletis blancardella, Lithophane antennata, Llattia octo (=Amyna axis), Lobesia botrana, Lophocampa spp., Loxagrotis albicosta, Loxostege spp. such as L. sticticalis, L. cereralis; Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanitis leda, Moccis spp. such as M. lapites, M. repanda; Mocis latipes, Monochroa fragariae, Mythimna separata, Nemapogon cloacella, Neoleucinodes elegantalls, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panolis flammea, Parnara spp., Papaipema nebris, Papilio cresphontes, Paramyelois transitella, Paranthrene regalis, Paysandisia archon, Pectinophora spp. such as P. gossypiella; Peridroma saucia, Perfleueoptera spp., such as P. coffeella; Phalera bucephala, Phryganidia californica, Phthorimaea spp. such as P. operculella; Phyllocnistis citrella, Phyllonorycter spp. such as P. blancardella, P. crataegella, P. issikii, P. ringoniella; Pieris spp. such as P. brassicae, P. rapae, P. napi; Pilocrocis tripunctata, Plathypena scabra, Platynota spp. such as P. flavedana, P. idaeusalis, P. stultana; Platyptilia carduidactyla, Plebejus argus, Plodia interpunctella, Plusla spp, Plutella maculipennis, Plutella xylostella, Pontia protoclica, Prays spp., Prodenia spp., Proxenus lepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subfiava, Sitotroga cerealella, Sparganothis pilleriana, Spilonota lechriaspis, S. ocellana, Spodoptera (=Lamphygma) spp. such as S. efidania, S. exigua, S. frugiperda, S. latisfascia, S. littoralis, S. litura, S. omithogalli; Stigmella spp., Stomopteryx subsecivella, Strymon bazochii, Sylepta derogata, Synanthedon spp. such as S. exitiosa, Tecia solanivora, Telehin licus, Thaumatopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp. such as T. cloacella, T. pellionella, Tineola bisselliella, Tortrix spp. such as T. viridana; Trichophaga tapetzella, Trichoplusla spp. such as T. ni; Tuta (=Scrobipalpula) absoluta, Udea spp. such as U. rubigalls, U. rubigalls; Virachola spp., Yponomeuta padella, and Zeiraphera canadensis; insects from the order of Coleoptera, for example Acalymma vittatum, Acanthoscehdes obtectus, Adoretus spp., Agelastica alai, Agrilus spp. such as A. anxius, A. planipennis, A. sinuatus; Agriotes spp. such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp. such as A. glabripennis; Anthonomus spp. such as A. eugenii, A. grandis, A. pomorum; Anthrenus spp., Aphthona euphoridae, Apion spp., Apogonia spp., Athous haemorrhoidals, Atomaria spp. such as A. linearis; Attagenus spp., Aulacophora femoralis, Blastophagus piniperda, Blitophaga undata, Bruchidius obtectus, Bruchus spp. such as B. lentis, B. pisorum, B. rufimanus; Byctiscus betulae, Calflagellum rufipenne, Callopistria fiondensis, Callosobruchus chinensis, Cameraria ohridella, Cassida nebulosa, Cerotoma trifurcate, Cetonia aurata, Ceuthorhynchus spp. such as C. assimilis, C. napi; Chaetocnema tibialis, Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp., Dactylispa balyi, Dectes texanus, Dermestes spp., Diabrotica spp. such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. simllaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica; Hypomeces squamosus, Hypothenemus spp., Ips typographus, Lachnosterna consanguinea, Lasioderma serricome, Latheticus oryzae, Lathridius spp., Lema spp. such as L. bilineata, L. melanopus; Leptinotarsa spp. such as L. decemlineata; Leptispa pygmaea, Limonius californicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp., Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyre; Phyllopertha horticola, Phyllophaga spp. such as P. helleri; Phyllotreta spp. such as P. chrysocephala, P. nemorum, P striolata, P vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophilus spp. such as S. granaria, S. oryzae, S. zeamais; Sphenophorus spp. such as S. levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T castaneum; Trogoderma spp., Tychius spp., Xylotrechus spp. such as X. pyrrhoderus; and, Zabrus spp. such as Z. tenebriodes; insects from the order of Diptera for example Aedes spp. such as A. aegypti, A. albopictus, A. vexans; Anastrepha ludens, Anopheles spp. such as A. albimanus, A. cruclans, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus; Culicoides furens, Culiseta inornata, CuAseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzukii, Fannia spp. such as F. canicularis; Gastraphilus spp. such as G. intestinails; Geomyza tipunctata, Glossina spp. such as G. fuscipes, G. morsilans, G. palpalis, G. tachinoides; Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprin, L. cuprina, L. sericata; Lycoria pectoralis, Mansonia titillanus, Mayetiola spp. such as M. destructor; Musca spp. such as M. autumnalis, M. domestica; Muscina stabulans, Oestrus spp. such as O. ovis; Opomyza forum, Oscinella spp. such as O. frit; Orseolia oryzae, Pegomya hysocyami, Phlebotomus argentipes, Phorbia spp. such as P. antiqua, P. brassicae, P. coarctata; Phytomyza gymnostoma, Prosimulium mixtum, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalis; Simulium viltatum, Silodiplosi:s mosellana, Stomoxys spp. such as S. calcitrans; Tabanus spp. such as T. atratus, T. bovinus, T. lineola, T. simllis; Tannia spp., Thecodiplosis japonensis, Tipula oleracea, Tipula paludosa, and Wohlfahrtia spp; insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. trilici; Heliothrips spp., Hercinothrips femoralis, Kakothfips spp., Microcephalothrips abdominalis, Neohydatothrips samayunkur, Pezothrips kellyanus, Rhipiphorothrips cruentatus, Scirtothrips spp. such as S. citri, S. dorsalis, S. perseae; Stenchaetothrips spp, Taeniothrilas cardamoni, Taeniothrips inconsequens, Thrips spp. such as T. imagines, T. hawaiiensis, T. oryzae, T. palmi, T. parvispinus, T. tabaci; insects from the order of Hemiptera for example, Acizzia jamatonica, Acrosternum spp. such as A. hilare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp. such as A. craccivora, A. fabae, A. forbesi, A. gossypii, A. grossulariae, A. maidiradicis, A. pomi, A. sambuci, A. schneideri, A. spiraecola; Arboridia apicalis, Arilus crilatus, Aspidiella spp., Aspidiotus spp., Atanus spp., Aulacaspis yasumatsui, Aulacorthum solani, Bactericera cockerelli (Paratrioza cockerelli), Bemisia spp. such as B. argentifolii, B. tabaci (Aleurodes tabaci); Blissus spp. such as B. leucopterus; Brachycaudus spp. such as B. carclui, B. helichrysi, B. persicae, B. prunicola; Brachycolus spp., Brachycorynella asparagi, Brevicoryne brassicae, Cacopsylla spp. such as C. fulguralis, C. pyricola (Psylla pin); Calligypona marginata, Calocoris spp., Campylomma livida, Capllophorus horni, Carneocephala fulgida, Cavelerius spp., Ceraplastes spp., Ceratovacuna lanigera, Ceroplastes cerfferus, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopelgs notatus, Dalbulus spp., Dasynus pipers, Dialeurodes spp. such as D. citrifolii; Dalbulus maidis, Diaphorina spp. such as D. citri; Diaspis spp. such as D. bromeliae; Dichelops furcatus, Diconocoris hewetti, Doralis spp., Dreyfusia nordmannianae, Dreyfusia piceae, Drosicha spp., Dysaphis spp. such as D. plantaginea, D. pyri, D. radicola; Dysaulacorthum pseudosolani, Dysdercus spp. such as D. cingulatus, D. intermedius; Dysmicoccus spp., Edessa spp., Geocorls spp., Empoasca spp. such as E. fabae, E. solana; Epidiaspis leperii, Eriosoma spp. such as E. lanigerum, E. pyricola; Erythroneura spp., Eurygaster spp. such as E. integriceps; Eusceks bilobatus, Euschistus spp. such as E. heros, E. impictiventris, E. servus; Fiornia theae, Geococcus coffeae, Glycaspis brimblecombei, Halyomorpha spp. such as H. halys; Heliopeltis spp., Homalodisca vitripennis (=H. coagulata), Horcias nobilellus, Hyalopterus pruni, Hyperomyzus lactucae, Icerya spp. such as I. purchase; Idiocerus spp., Idloscopus spp., Laodelphax striatellus, Lecanium spp., Lecanoideus floccissimus, Lepidosaphes spp. such as L. ulmi; Leptocorisa spp., Leptoglossus phyllopus, Lipaphis erysimi, Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadrilineatus, Mahanarva fimbriolata, Megacopta cribraria, Megoura viciae, Melanaphis pyrarius, Melanaphis sacchari, Melanocallis (=Tinocallis) caryaefoliae, Metcalfiella spp., Metopolophium dirhodum, Monellia costals, Monelliopstis pecanis, Myzocallis coryli, Murgantia spp., Myzus spp. such as M. ascalonicus, M. cerasi, M. nicotianae, M. persicae, M. varians; Nasonovia ribis-nigri, Neotoxoptera formosana, Neomegalotomus spp, Nephotettix spp. such as N. malayanus, N. nigroplctus, N. parvus, N. virescens; Nezara spp. such as N. virdula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinii, Phorodon humuli, Phylloxera spp. such as P. devastatrix, Piesma quadrata, Piezodorus spp. such as P. guildinii; Pinnaspis aspidistrae, Planococcus spp. such as P. citri, P. ficus; Prosapia bicincta, Protopulvinaria pyriformis, Psallus seriatus, Pseudacysta persea, Pseudaulacaspis pentagona, Pseudococcus spp. such as P. comstocki; Psylla spp. such as P. mali; Pteromalus spp., Pulvinaria amygdall, Pyrilla spp., Quadraspidiotus spp., such as Q. perniciosus; Quesada gigas, Rastrococcus spp., Reduvius senilis, Rhizoecus americanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis mali, Scaptocoris spp., Scaphoides titanus, Schlzaphis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina), Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp. such as T. aurantll; Trialeurodes spp. such as T. abutilonea, T. ricin, T. vaporariorum; Triatoma spp., Trioza spp., Typhlocyba spp., Unaspis spp. such as U. citri, U. yanonensis; and Viteus vitifolii; insects from the order Hymenoptera for example Acanthomyops interjectus, Athalia rosae, Atta spp. such as A. capiguara, A. cephalotes, A. cephalotes, A. laevigata, A. robusta, A. sexdens, A. texana, Bombus spp., Brachymyrmex spp., Camponotus spp. such as C. floridanus, C. pennsylvanicus, C. modoc; Cardibcondyla nuda, Chalibion sp, Crematogaster spp., Dasymutilla occidentalis, Diprion spp., Dolichovespula maculata, Dorymyrmex spp., Dryocosmus kuriphilus, Formica spp., Hoplocampa spp. such as H. minuta, H. testudinea; Iridomyrmex humilis, Lasius spp. such as L. niger, Linepithema humile, Liometopum spp., Leptocybe invasa, Monomorium spp. such as M. pharaonis, Monomorium, Nylandria fulva, Pachycondyla chinensis, Paratrechina longicornis, Paravespula spp., such as P. germanica, P. pennsylvanica, P. vulgaris; Pheidole spp. such as P. megacephala; Pogonomyrmex spp. such as P. barbatus, P. californicus, Polistes rubiginosa, Prenolepis impairs, Pseudomyrmex gracilis, Schelipron spp., Sirex cyaneus, Solenopsis spp. such as S. geminata, S. invicta, S. molesta, S. richteri, S. xylow, Sphecius speciosus, Sphex spp., Tapinoma spp. such as T. melanocephalum, T sessile; Tetramorium spp. such as T. caespitum, T. bicarinatum, Vespa spp. such as V. crabro; Vespula spp. such as V. squamosal; Wasmannia auropunctata, Xylocopa sp; nsects from the order Orthoptera for example Acheta domesticus, Calliptamus italicus, Chortoicetes terminifera, Ceuthophilus spp., Diastrammena asynamora, Dociostaurus maroccanus, Gryllotalpa spp. such as G. africana, G. gryllotalpa; Gryllus spp., Hieroglyphus daganensis, Kraussaria angulifera, Locusta spp. such as L. migratoria, L. pardalina; Melanoplus spp. such as M. bivittatus, M. femurrubrum, M. mexicanus, M. sanguinipes, M. spretus; Nomadacris septemfasciata, Oedaleus senegalensis, Scapteriscus spp., Schistocerca spp. such as S. americana, S. gregaria, Stemopelmatus spp., Tachycines asynamorus, and Zonozerus variegatus; pests from the Class Arachnida for example Acari,e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentor spp. such as D. silvarum, D. andersoni, D. variabilis, Hyalomma spp. such as H. truncatum, Ixodes spp. such as I. ricinus, I. rubicundus, I. scapularis, I. holocyclus, I. pacificus, Rhipicephalus sanguineus, Ornithodorus spp. such as O. moubata, O. hermsi, O. turicata, Ornithonyssus bacoti, Otobius megnini, Dermanyssus gallinae, Psoroptes spp. such as P. ovis, Rhipicephalus spp. such as R. sanguineus, R. appendiculatus, Rhipicephalus evertsi, Rhizoglyphus spp., Sarcoptes spp. such as S. Scabier; and Family Eriophyidae including Aceria spp. such as A. sheldoni, A. anthocoptes, Acallitus spp., Aculops spp. such as A. lycopersici, A. pelekassi; Aculus spp. such as A. schlechtendali; Colomerus vitis, Epitrimerus pyri, Phyllocoptruta oleivora; Eriophytes ribis and Eriophyes spp. such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenicis; Family Tetranychidae including Eotetranychus spp., Eutetranychus spp., Oligonychus spp., Petrobia latens, Tetranychus spp. such as T. cinnabarinus, T. evansi, T. kanzawai, T. pacificus, T. phaseulus, T. telarius and T. urticae; Bryobia praetiosa; Panonychus spp. such as P. ulmi, P. citri; Metatetranychus spp. and Oligonychus spp. such as O. pratens/s, O. perseae, Vasates lycopersici; Raoiella indica, Family Carpoglyphidae including Carpoglyphus spp.; Penthaleidae spp. such as Halotydeus destructor, Family Demodicidae with species such as Demodex spp.; Family Trombicidea including Trombicula spp.; Family Macronyssidae including Ornothonyssus spp.; Family Pyemotidae including Pyemotes tritict, Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa; pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifolii; Seed gall nematodes, Anguina spp.; Stem and foliar nematodes, Aphelenchoides spp. such as A. besseyi; Sting nematodes, Belonolaimus spp. such as B. longicaudatus; Pine nematodes, Bursaphelenchus spp. such as B. lignicolus, B. xylophilus; Ring nematodes, Criconema spp., Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemodes spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. claytoni, T. dubius; Citrus nematodes, Tylenchulus spp. such as T. semipenetrans; Dagger nematodes, Xiphinema spp.; and other plant parasitic nematode species; insects from the order Isoptera for example Calotermes flavicollis, Coptotermes spp. such as C. formosanus, C. gestroi, C. acinaciformis; Cornitermes cumulans, Cryptotermes spp. such as C. brevis, C. cavifrons; Globliermes sulfureus, Heterotermes spp. such as H. aureus, H. longiceps, H. tenuis; Leucotermes fiavipes, Odontotermes spp., Incisitermes spp. such as I. minor, I. Snyder, Marginitermes hubbardi, Mastotermes spp. such as M. darwiniensis Neocapritermes spp. such as N. opacus, N. parvus; Neotermes spp., Procornitermes spp., Zootermopsis spp. such as Z. angusticollis, Z. nevadensis, Reticulliermes spp. such as R. hesperus, R. tibialis, R. speratus, R. flavipes, R. grassei, R. lucifugus, R. santonensis, R. virginicus; Termes natalensis; insects from the order Blattaria for example Blatta spp. such as B. orientalis, B. lateralis; Blattella spp. such as B. asahinae, B. germanica; Leucophaea maderae, Panchlora nivea, Periplaneta spp. such as P. americana, P. australasiae, P. brunnea, P. fuligginosa, P. japonica; Supella longipalpa, Parcoblatta pennsylvanica, Eurycotis floridana, Pycnoscelus surinamensis; insects from the order Siphonoptera for example Cediopsylla simples, Ceratophyllus spp., Ctenocephalides spp. such as C. fells, C. canis, Xenopsylla cheopis, Pulex irritans, Trichodectes canis, Tunga penetrans, and Nosopsyllus fasciatus; insects from the order Thysanura for example Lepisma saccharina, Ctenolepisma urbana, and Thermobia domestica, pests from the class Chilopoda for example Geophilus spp., Scutigera spp. such as Scutigera coleoptrata; pests from the class Diptopoda for example Blaniulus guttulatus, Julus spp., Narceus spp.; pests from the class Symphyla for example Scutigerella immaculata; insects from the order Dermaptera, for example Forficula auricularia; insects from the order Collembola, for example Onychiurus spp., such as Onychiurus armatus; pests from the order Isopoda for example, Armadillidium vulgare, Oniscus asellus, Porcellio scaber, insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp. Examples of further pest species which may be controlled by compounds of fomula (I) include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori Bunostomum spp., Chabertia spp., Clonorchis spp., Cooperla spp., acrocoelium spp., Dictyocaulus filaria, Diphyllobothrium latum, Dracunculus medinensls, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchls spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichurla, Wuchereria bancrofti.

In one embodiment, the methods and uses of the application relate to plants that carry a specific trait. The term “trait” relates to a specific feature, or a combination of features of a plant that have been added either by conventional breeding, i.e. a plant, which has not been modified by mutagenesis, or genetic engineering, or to features of a plant that have been added by mutagenesis or genetic engineering. This feature, or the combination of features, are described in comparison to respective non-modified control plants, i.e. plants that were not subjected to procedures for producing a plant that carries the same trait. The term “trait” in its singular, as used in this application, also refers to combinations of traits. Such traits may enable a cotton plant to be resistant to active ingredients, such as herbicides, or to produce biologically active compounds that control infestation by plant pests. Examples of traits are better plant growth, increased tolerance to high or low temperatures, increased tolerance to drought, or to water or soil salt content, increased flowering performance, easier harvesting, accelerated maturation, higher harvest yields, higher quality and/or a higher nutritional value of the harvested products, better storage stability and/or processability of the harvested products.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected. Further and particularly emphasized examples of such traits are a better defense of the plants against animal and microbial pests, such as against insects, mites, phytopathogenic fungi, bacteria and/or viruses, and also increased tolerance of the plants to certain herbicidal active compounds. Preferred traits are insect resistance and herbicide tolerance, more preferably insect resistance, in particular resistance against Coleopterans, Hemipterans, and Lepidopterans. In one embodiment, the trait is a resistance against Coleopterans. Usually, the methods and uses of the application relate to cotton plants, which are commercially available. Depending on the cotton plant and its traits, its location and growth conditions (soils, climate, vegetation period, diet), the disclosed uses and methods of application may also result in superadditive (“synergistic”) effects. Examples of genetically modified cotton plants, as well of traits, and modified and/or incorporated genes are listed in Table B. Single rows and combinations of rows are each embodiments of for the methods and uses of the invention. Cotton plants that were assigned with a specific tradename are commercially available. All other can be produced by the skilled person based on the Event Code by known methods, or can be obtained from deposition facilities of biological material.

TABLE B
Events, traits, genes, and developing companies of cotton plants and/or propagation
material. Explanations for abbreviations are listed in Table C.

| | | | | | Developing/ |
|---|---|---|---|---|---|
| | | | | Trait (Trait | Producing |
| No | Event Name | Event Code | Tradename | type)/Gene | Company |
| 1 | 19-51a | DD-Ø1951A-7 | — | HT (SU)/S4-hrA | Dupont |
| 2 | 281-24-236 | DAS-24236-5 | — | IR (BL)/cry1F | Dow |
| 3 | 281-24-236 × | DAS-24236-5 × | WideStrike ™ | IR (BL)/cry1F | Dow |
| | 3006-210-23 | DAS-21Ø23-5 | Cotton | IR (BL)/cry1Ac | |
| | (MXB-13) | | | | |
| 4 | 3006-210-23 | DAS-21Ø23-5 | — | IR (BL)/Cry1Ac | Dow |
| 5 | 3006-210-23 × | DAS-21Ø23-5 × | WideStrike ™ | HT (Gly)/cp4 epsps | Monsanto |
| | 281-24-236 × | DAS-24236-5 × | Roundup | (aroA:CP4) | |
| | MON1445 | MON-Ø1445-2 | Ready ™ | IR (BL)/cry1F | |
| | | | Cotton | IR (BL)/cry1Ac | |
| | | | | HT (Glu)/bar | |
| 6 | 3006-210-23 × | DAS-21Ø23-5 × | Widestrike ™ | HT (Gly)/cp4 epsps | Dow & Monsanto |
| | 281-24-236 × | DAS-24236-5 × | Roundup | (aroA:CP4) | |
| | MON88913 | MON-88913-8 | Ready Flex ™ | IR (BL)/cry1F | |
| | | | Cotton | IR (BL)/cry1Ac | |
| | | | | HT (Glu)/bar | |
| 7 | 3006-210-23 × | DAS-21Ø23-5 × | Widestrike ™ × | IR (BL)/cry1Ac | Dow |
| | 281-24-236 × | DAS-24236-5 × | Roundup | IR (BL)/vip3A(a) | |
| | MON88913 × | MON-88913-8 × | Ready Flex ™ × | IR (BL)/cry1F | |
| | COT102 | SYN-IR1Ø2-7 | VIPCOT ™ | HT (Gly)/cp4 epsps | |
| | | | Cotton | (aroA:CP4) | |
| | | | | HT (Glu)/pat | |
| 8 | 31707 | — | BXN ™ Plus | HT (Ox)/bxn | Monsanto |
| | | | Bollgard ™ | IR (BL)/cry1Ac | |
| | | | Cotton | | |
| 9 | 31803 | — | BXN ™ Plus | HT (Ox)/bxn | Monsanto |
| | | | Bollgard ™ | IR (BL)/cry1Ac | |
| | | | Cotton | | |
| 10 | 31807 × | — | — | HT (Ox)/bxn | Monsanto |
| | 31808 | | | IR (BL)/cry1Ac | |
| 11 | 31807 | — | BXN ™ Plus | HT (Ox)/bxn | Monsanto |
| | | | Bollgard ™ | IR (BL)/cry1Ac | |
| | | | Cotton | | |
| 12 | 31808 | — | BXN ™ Plus | HT (Ox)/bxn | Monsanto |
| | | | Bollgard ™ | IR (BL)/cry1Ac | |
| | | | Cotton | | |
| 13 | 42317 | — | BXN ™ Plus | HT (Ox)/bxn | Monsanto |
| | | | Bollgard ™ | IR (BL)/cry1Ac | |
| | | | Cotton | | |
| 14 | BNLA-601 | — | — | IR (BL)/cry1Ac | CICR and UASD |
| | | | | | (India) |
| 15 | BXN10211 | BXN-1Ø211-9 | BXN ™ Cotton | HT (Ox)/bxn | Monsanto |
| | (10211) | | | | |
| 16 | BXN10215 | BXN-1Ø215-4 | BXN ™ Cotton | HT (Ox)/bxn | Monsanto |
| | (10215) | | | | |
| 17 | BXN10222 | BXN-1Ø222-2 | BXN ™ Cotton | HT (Ox)/bxn | Monsanto |
| | (10222) | | | | |
| 18 | BXN10224 | BXN-1Ø224-4 | BXN ™ Cotton | HT (Ox)/bxn | Monsanto |
| | (10224) | | | | |
| 19 | COT102 | SYN-IR1Ø2-7 | VIPCOT ™ | IR (BL)/vip3A(a) | Syngenta |
| | (IR102) | | Cotton | | |
| 20 | COT102 × | SYN-IR1Ø2-7 × | VIPCOT ™ | IR (BL)/vip3A(a) | Syngenta |
| | COT67B | SYN-IR67B-1 | Cotton | IR (BL)/cry1Ab | |
| 21 | COT102 × | SYN-IR1Ø2-7 × | VIPCOT ™ | IR (BL)/vip3A(a) | Syngenta |
| | COT67B × | SYN-IR67B-1 × | Roundup | IR (BL)/cry1Ab | |
| | MON88913 | MON-88913-8 | Ready Flex ™ | | |
| | | | Cotton | | |
| 22 | COT102 × | SYN-IR1Ø2-7 × | Bollgard ® III | IR (BL)/vip3A(a) | Monsanto |
| | MON15985 | MON-15985-7 | | IR (BL)/cry1Ac | |
| | | | | IR (BL)/cry2Ab2 | |
| 23 | COT102 × | SYN-IR1Ø2-7 × | Bollgard ® III × | IR (BL)/vip3A(a) | Monsanto |
| | MON15985 × | MON-15985-7 × | Roundup | IR (BL)/cry1Ac | |
| | MON88913 | MON-88913-8 | Ready ™ | IR (BL)/cry2Ab2 | |
| | | | Flex ™ | HT (Gly)/cp4 epsps | |
| | | | | (aroA:CP4) | |
| 24 | COT67B | SYN-IR67B-1 | — | IR (BL)/cry1Ab | Syngenta |
| | (IR67B) | | | | |
| 25 | Event1 | | JK 1 | IR (BL)/cry1Ac | JK Agri |
| | | | | | Genetics |
| | | | | | Ltd. (India) |
| 26 | GFM Cry1A | GTL-GFM311-7 | — | IR (BL)/cry1Ab-Ac | Nath Seeds/ |
| | | | | | Global Trans- |
| | | | | | genes Ltd |
| | | | | | (India) |
| 27 | GHB119 | BCS-GHØØ5-8 | — | HT (Glu)/bar | Bayer Crop |
| | | | | IR (BL)/cry2Ae | Science |
| 28 | GHB614 | BCS-GHØØ2-5 | GlyTol ™ | HT (Gly)/2mepsps | Bayer Crop |
| | | | | | Science |
| 29 | GHB614 × | BCS-GHØØ2-5 × | GlyTol ™ | HT (Gly)/2mepsps | Bayer Crop |
| | LLCotton25 | ACS-GHØØ1-3 | Liberty Link ™ | HT (Glu)/bar | Science |
| 30 | GHB614 × | BCS-GHØØ2-5 × | — | HT (Gly)/2mepsps | Bayer Crop |
| | LLCotton25 × | ACS-GHØØ1-3 × | | HT (Glu)/bar | Science |
| | MON15985 | MON-15985-7 | | IR (BL)/cry1Ac | |
| | | | | IR (BL)/cry2Ab2 | |
| 31 | GHB614 × | BCS-GHØØ2-5 × | — | HT (Gly)/2mepsps | Bayer Crop |
| | MON15985 | MON-15985-7 | | IR (BL)/cry1Ac | Science |
| | | | | IR (BL)/cry2Ab2 | |
| 32 | GHB614 × | BCS-GHØØ2-5 × | Glytol ™ × | HT (Gly)/2mepsps | Bayer Crop |
| | T304-40 × | BCS-GHØØ4-7 × | Twinlink ™ | HT (Glu)/bar | Science |
| | GHB119 | BCS-GHØØ5-8 | | IR (BL)/cry1Ab | |
| | | | | IR (BL)/cry2Ae | |
| 33 | GK12 | | — | IR (BL)/cry1Ab-Ac | Chinese Academy |
| | | | | | of Agricultural |
| | | | | | Sciences |
| 34 | LLCotton25 | ACS-GHØØ1-3 | Fibermax ™ | HT (Glu)/bar | Bayer Crop |
| | | | Liberty Link ™ | | Science |
| 35 | LLCotton25 × | ACS-GHØØ1-3 × | Fibermax ™ | HT (Glu)/bar | — |
| | MON15985 | MON-15985-7 | Liberty Link ™ | IR (BL)/cry1Ac | |
| | | | Bollgard II ™ | IR (BL)/cry2Ab2 | |
| 36 | MLS 9124 | — | — | IR (BL)/cry1C | Metahelix Life |
| | | | | | Sciences Pvt. |
| | | | | | Ltd (India) |
| 37 | MON1076 | MON-89924-2 | Bollgard ™ | IR (BL)/cry1Ac | Monsanto |
| | | | Cotton | | |
| 38 | MON1445 | MON-Ø1445-2 | Roundup | HT (Gly)/cp4 epsps | Monsanto |
| | | | Ready ™ | (aroA:CP4) | |
| | | | Cotton | | |
| 39 | MON15985 | MON-15985-7 | Bollgard II ™ | IR (BL)/cry1Ac | Monsanto |
| | | | Cotton | IR (BL)/cry2Ab2 | |
| 40 | MON15985 × | MON-15985-7 × | Roundup | HT (Gly)/cp4 epsps | Monsanto |
| | MON1445 | MON-Ø1445-2 | Ready ™ | (aroA:CP4) | |
| | | | Bollgard II ™ | IR (BL)/cry1Ac | |
| | | | Cotton | IR (BL)/cry2Ab2 | |
| 41 | MON1698 | MON-89383-1 | Roundup | HT (Gly)/cp4 epsps | Monsanto |
| | | | Ready ™ | (aroA:CP4) | |
| | | | Cotton | | |
| 42 | MON531 | MON-ØØ531-6 | Bollgard ™ | IR (BL)/cry1Ac | Monsanto |
| | | | Cotton, | | |
| | | | Ingard ™ | | |
| 43 | MON531 × | MON-Ø531-6 × | Roundup | HT (Gly)/cp4 epsps | Monsanto |
| | MON1445 | MON-Ø1445-2 | Ready ™ | (aroA:CP4) | |
| | | | Bollgard ™ | IR (BL)/cry1Ac | |
| | | | Cotton | | |
| 44 | MON757 | MON-ØØ757-7 | Bollgard ™ | IR (BL)/cry1Ac | Monsanto |
| | | | Cotton | | |
| 45 | MON88913 | MON-88913-8 | Roundup | HT (Gly)/cp4 epsps | Monsanto |
| | | | Ready ™ | (aroA:CP4) | |
| | | | Flex ™ | | |
| | | | Cotton | | |
| 46 | MON88913 × | MON-88913-8 × | Roundup | HT (Gly)/cp4 epsps | Monsanto |
| | MON15985 | MON-15985-7 | Ready ™ | (aroA:CP4) | |
| | | | Flex ™ | IR (BL)/cry1Ac | |
| | | | Bollgard II ™ | IR (BL)/cry2Ab2 | |
| | | | Cotton | | |
| 47 | Ngwe Chi 6 Bt | — | Ngwe Chi 6 Bt | — | Cotton and |
| | | | | | Sericulture |
| | | | | | Department |
| | | | | | (Myanmar) |
| 48 | SGK321 | — | — | IR (BL)/cry1A | Chinese Academy |
| | | | | IR (BRun)/CpTI | of Agricultural |
| | | | | | Sciences |
| 49 | T303-3 | BCS-GHØØ3-6 | — | IR (BL)/cry1Ab | Bayer Crop |
| | | | | HT (Glu)/bar | Science |
| 50 | T304-40 | BCS-GHØØ4-7 | — | IR (BL)/cry1Ab | Bayer Crop |
| | | | | HT (Glu)/bar | Science |
| 51 | T304-40 × | BCS-GHØØ4-7 × | TwinLink ™ | IR (BL)/cry1Ab | Bayer Crop |
| | GHB119 | BCS-GHØØ5-8 | Cotton | HT (Glu)/bar | Science |
| | | | | IR (BL)/cry2Ae | |
| 52 | 81910 | DAS-81910-7 | — | HT (2,4-D)/aad-12 | Dow |
| | | | | HT (Glu)/pat | |
| 53 | MON88701 | MON 887Ø1-3 | — | HT (Dic)/dmo | Monsanto |
| | | | | HT (Glu)/bar | |
| 54 | MON88701 × | MON 887Ø1-3 × | — | HT (Dic)/dmo | Monsanto |
| | MON88913 | MON-88913-8 | | HT (Glu)/bar | |
| | | | | HT (Gly)/cp4 epsps | |
| | | | | (aroA:CP4) | |
| 55 | GHB614 × | BCS-GHØØ2-5 × | Glytol ™ × | HT (Glu)/bar | Bayer Crop |
| | T304-40 × | BCS-GHØØ4-7 × | Twinlink ™ × | IR (BL)/cry1Ab | Science |
| | GHB119 × | BCS-GHØØ5-8 × | VIPCOT ™ | IR (BL)/cry2Ae | |
| | COT102 | SYN-IR1Ø2-7 | Cotton | IR (BL)/vip3A(a) | |
| | | | | HT (Gly)/2mepsps | |
| 56 | MON88701 × | MON 887Ø1-3 × | — | HT (Glu)/bar | Monsanto |
| | MON88913 × | MON-88913-8 × | | HT (Dic)/dmo | |
| | MON15985 | MON-15985-7 | | HT (Gly)/cp4 epsps | |
| | | | | (aroA:CP4) | |
| | | | | IR (BL)/cry1Ac | |
| | | | | IR (BL)/cry2Ab2 | |
| 57 | 281-24-236 × | DAS-24236-5 × | — | IR (BL)/cry1Ac | Dow |
| | 3006-210-23 × | DAS-21023-5 × | | IR (BL)/vip3A(a) | |
| | COT102 | SYNIR102-7 | | IR (BL)/cry1F | |
| | | | | HT (Gly)/cp4 epsps | |
| | | | | (aroA:CP4) | |
| | | | | HT (Glu)/pat | |

TABLE C
explanations for abbreviations in Table B

| Trait | Trait - full name | Trait type | Trait type - full name |
|---|---|---|---|
| HT | Herbicide Tolerance | HT (Gly) | glyphosate tolerance |
| | | HT (Glu) | glufosinate tolerance |
| | | HT (2,4-D) | resistance against 2,4-D |
| | | | Choline |
| | | HT (SU) | sulfonylurea tolerance |
| | | HT (Ox) | oxynil herbicide tolerance |
| | | | (e.g. bromoxynil) |
| | | HT (Dic) | dicamba tolerance |
| IR | Insect resistance | IR (BL) | broad spectrum resistance |
| | (including | | against lepidopterans |
| | Nematodes ) | | (above ground worms) |
| | | IR (BRun) | broad range resistance, |
| | | | not further specified |

Detailed information of the cotton plants listed in Table B is available online under www.isaaa.org. Traits that are emphasized in particular are the increased defense of the plants against insects, arachnids, nematodes, slugs and snails, preferably against insects, by virtue of toxins formed in the plants, in particular those formed in the plants by expression of genes of Bacillus thuringiensis (for example by the genes cry1A, cry1Aa, cry1Ab, cry1Ac, cry2A, cry3A, cry3B2, cry9c, cry2Ab, cry3Bb cry1F, and vip3Aa20), or of genes derived from Bacillus thuringiensis by mutagenesis or genetic engineering (for examples the genes ecry3, mcry3A, mocry1F) (referred to herein as “Bt plants”), or by expression of other genes known to convey insect resistance, such as CpTI (Cowpea trypsin inhibitor gene), or by combinations of these genes. In one embodiment, insect resistance is provided by at least one gene of Bacillus thuringiensis. In another embodiment, insect resistance is provided by a CpTI gene. In another embodiment, insect resistance is provided by at least one gene derived from a Bacillus thuringiensis gene by mutagenesis or genetic engineering. p In one embodiment, the gene derived from the Bacillus thuringiensis gene may have at least one base mutation that causes at least on amino acid exchange, wherein both exchanges are in comparison with the respective wild type sequence of the gene, or protein. In another embodiment, the gene derived from a Bacillus thuringiensis gene may be truncated with regard to the wild type sequence by up to 30 base pairs, preferably by up to 150 base pairs, and most preferably by up to 500 base pairs. In one embodiment, the trait is conveyed by at least one gene selected from cry1A, cry1Ab, cry1Ac, cry1F, cry2A, cry2Ae, cry2Ab2, cry1Ab-Ac, cry3A, cry3B2, cry9c, cry2Ab, and cry3Bb, and combinations thereof. In another embodiment, the trait is conveyed by at least one gene selected from cry1A, cry1Ab, cry1Ac, cry1F, cry2Ae, cry2Ab2, cry1Ab-Ac. In another embodiment, the trait is conveyed by at least one gene selected from cry1A, cry1Ab, cry1Ac, cry1F, cry2Ae, cry2Ab2, cry1Ab-Ac, vip3A(a). In another embodiment, the trait is conveyed by at least one gene selected from cry1A, cry1Ab, cry1Ac, cry1C, cry1F, cry2Ae, cry2Ab2, cry1Ab-Ac, vip3A(a), and CpTI. In another embodiment, the trait is conveyed by at least one gene selected from cry1A, cry1Ab, cry1Ac, cry1C, cry1F, cry2Ae, cry2Ab2, cry1Ab-Ac, vip3A(a), and CpTI. In another embodiment, the trait is conveyed by at least one gene selected from cry1Ab, cry1Ac, cry1F, cry2Ab2, cry2Ae, vip3A(a), and combinations thereof. In another embodiment, the trait is conveyed by at least one gene selected from cry1Ab, cry1Ac, cry1F, cry2Ab2, and combinations thereof. In another embodiment, the trait is conveyed by at least one gene selected from cry1Ac, cry2Ab2, and the combination thereof. In another embodiment, the trait is conveyed by cry1F. In another embodiment, the trait is conveyed by cry1Ac. In another embodiment, the trait is conveyed by vip3A(a). In another embodiment, the trait is conveyed by cry2Ab2. In another embodiment, the trait is conveyed by cry1Ab-Ac. In another embodiment, the trait is conveyed by cry2Ae. In another embodiment, the trait is conveyed by cry1A. In another embodiment, the trait is conveyed by cry1Ab. In another embodi-ment, the trait is conveyed by CpTI.

Traits that are also particularly emphasized are the increased defense of the plants against fungi, bacteria and viruses by systemic acquired resistance, systemin, phytoalexins, elicitors and resistance genes and correspondingly expressed proteins and toxins. Traits that are furthermore particularly emphasized are the increased tolerance of the plants to certain herbicidally active compounds, for example glyphosate, glufosinate, 2,4-D Choline, sulfonylurea, dicamba, or oxinyl. Accordingly, the cotton plant may have a herbicide tolerance that is conveyed by at least one gene selected from aad-12, bar, bxn, cp4 epsps, dmo, 2mepsps, pat, S4-hrA, and combinations thereof. In one embodiment, the herbicide tolerance is conveyed by at least one gene selected from aad-12, bar, cp4 epsps, dmo, 2mepsps, pat, and combinations thereof, preferably bar. The genes which impart the desired traits in question can also be present in combination with one another in the transgenic plants.

Suitable genetically modified cotton are sold under the trade names Glytol™×Twinlink™×VIPCOT™ Cotton, Glytol™×Twinlink™×VIPCOT™ Cotton, TwinLink™ Cotton, Ngwe Chi 6 Bt, Roundup Ready™ FIex™ Bollgard II™ Cotton, Roundup Ready™ FIex™ Cotton, Bollgard™ Cotton, Fibermax™ Liberty Link™ Bollgard II™, Fibermax™ Liberty Link™, Glytol™×Twinlink™, GlyTol™ Liberty Link™, GlyTol™, JK 1, Bollgard® III×Roundup Ready™ FIex™, Bollgard® III, VIPCOT™ Roundup Ready FIex™ Cotton, VIPCOT™ Cotton, BXN™ Cotton, BXN™ Plus Bollgard™ Cotton, Widestrike™×Roundup Ready FIex™×VIPCOT™ Cotton, Widestrike™ Roundup Ready Flex™ Cotton, WideStrike™ Cotton, and WideStrike™ Roundup Ready™ Cotton. Preferred cotton cultivars are sold under the trade names of Bollgard™, Bollgard II™, Widestrike™, and TwinLink™, in particular Bollgard II™.

The plants usually have a genetic event name selected from 281-24-236, 3006-210-23, 31707, 31708, 31807, 31808, 31803, 42317, BNLA-601, COT67B, COT102, Event1, GHB119, GFM Cry1A, GK12, MLS 9124, MON1076, MON15985, MON531, MON757, SGK321, T303-3, T304-40, and combinations thereof, preferably 3006-210-23, 31707, 31708, 42317, BNLA-601, Event1, MON531, MON1076, MON15985, MON757, and combinations thereof, and most preferably MON15985.

The components of the ginkgo tree or the pesticidal compositions comprising them are used to control pests of a cotton plant by contacting the cotton plant, parts of it, or its plant propagation material, the pests, their food supply, habitat or breeding grounds with a pesticidally effective amount of components of the ginkgo tree. In one embodiment, application of the components of the ginkgo tree is usually on the cotton plant, parts of it, or its propagation material. In one embodiment, application of the components of the ginkgo tree is usually on the cotton plant. In another embodiment, application of the components of the ginkgo tree is usually on the propagation material of cotton plants. In another embodiment, application of the components of the ginkgo tree is usually to the locus of the cotton plant. Application may be before infestation or when the pest is present. Application of the components of the ginkgo tree can be performed according to any of the usual modes of application, e.g. foliar, drench, soil, in furrow, seed treatment etc. In one embodiment, the components of the ginkgo tree are applied by soil drench application. In another embodiment, the components of the ginkgo tree are applied for protecting plant propagation material, preferably by seed-treatment to seeds of cotton crops. In yet another embodiment, the components of the ginkgo tree are applied by in-furrow treatment. In yet another embodiment, the components of the ginkgo tree are applied by foliar application. The components of the ginkgo tree may be applied in combination with an attractant. An attractant is a chemical that causes the insect to migrate towards the location of application. For control of stinkbugs it can be advantageous to apply the components of the ginkgo tree with an attractant, particularly when the application is foliar. Pentatomidae are often located near to the ground, and application of an attractant may encourage migration up the plant towards the active ingredient. Suitable attractants include glucose, sacchrose, salt, glutamate, citric acid, soybean oil, peanut oil and soybean milk. Glutamate and citric acid are of particular interest, with citric acid being preferred. An attractant may be premixed with the components of the ginkgo tree prior to application, e.g. as a readymix or tankmix, or by simultaneous application or sequential application to the plant. Suitable rates of attractants are for example 0.02 kg/ha-3 kg/ha. The components of the ginkgo tree are preferably used for pest control at 1-500 g/ha, preferably 10-100 g/ha.

The components of the ginkgo tree may be applied in the methods of the present invention in mixtures with fertilizers (for example nitrogen-, potassium- or phosphorus-containing fertilizers). Suitable formulation types include granules of fertilizer. The mixtures preferably contain up to 25% by weight of the compound of the invention.

The components of the ginkgo tree may be mixed with other compounds having biological activity, for example micronutrients or compounds having fungicidal activity or which possess plant growth regulating, herbicidal, insecticidal, nematicidal or acaricidal activity.

The components of the ginkgo tree applied in the uses and methods of application of the present invention may be the sole active ingredient of the composition or it may be admixed with one or more additional active ingredients such as an insecticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the components of the ginkgo tree; or help to overcome or prevent the development of resistance to individual components. The particular additional active ingredient will depend upon the intended utility of the composition. According to one embodiment of the present invention, individual components of the composition according to the invention such as parts of a kit or parts of a binary or ternary mixture may be mixed by the user himself in a spray tank and further auxiliaries may be added, if appropriate. The components of the ginkgo tree may be mixed with soil, peat or other rooting media for the protection of plants against seed-borne, soil-borne or foliar fungal diseases. Examples of suitable synergists for use in the compositions include piperonyl butoxide, sesamex, safroxan and dodecyl imidazole. Suitable herbicides and plant-growth regulators for inclusion in the compositions will depend upon the intended target and the effect required. Preferably the components of the ginkgo tree are mixed with a herbicide. Examples of herbicides are glyphosate, gluphosinate, sulfonylurea, imidazoline, or a HPPD inhibitor.

The components of the ginkgo tree may be present in form of agrochemical compositions comprising one or more auxiliary agents and at least one component of the ginkgo tree and/or one of its individual embodiments, which are applied in the methods of the present invention. An agrochemical composition comprises a pesticidally effective amount of the components of the ginkgo tree and/or one of its individual embodiments. An agrochemical composition comprises a pesticidally effective amount of the component of the ginkgo tree. The term “pesticidally effective amount” is defined below.

The components of the ginkgo tree can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders, or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. Suitable auxiliaries are solvents, liquid carriers, solid carriers, or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders. Suitable solvents and liquid carriers are water and organic solvents, such as mineral oil fractions of medium to high boiling point, e.g. kerosene, diesel oil; oils of vegetable, or animal origin; aliphatic, cyclic and aromatic hydrocarbons, e. g. toluene, paraffin, tetrahydronaphthalene, alkylated naphthalenes; alcohols, e.g. ethanol, propanol, butanol, benzyl alcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers, or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol.1: Emulsifiers & Detergents, McCutcheon's Directories, Glen Rock, USA, 2008 (International Ed. or North American Ed.)

Suitable anionic surfactants are alkali, alkaline earth or ammonium salts of sulfonates, sulfates, phosphates, carboxylates, and mixtures thereof. Examples of sulfonates are alkylaryl sulfonates, diphenylsulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates. Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters that have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpoly-glucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a negligible or even no pesticidal activity themselves, and which improve the biological performance of the components of the ginkgo tree on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates. Suitable bactericides are bronopol and isothiazolinone derivatives such as alkylisothiazolinones and benzisothiazolinones.

Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable tackifiers or binders are polyvinylpyrrolidons, polyvinylacetates, polyvinylalcohols, polyacrylates, biological or synthetic waxes, and cellulose ethers.

Examples for composition types and their preparation are:
i) Water-soluble concentrates (SL, LS): 10-60 wt % of a component of the ginkgo tree and 5-15 wt % wetting agent (e.g. alcohol alkoxylates) are dissolved in water and/or in a water-soluble solvent (e.g. alcohols) up to 100 wt %. The active substance dissolves upon dilution with water.
ii) Dispersible concentrates (DC): 5-25 wt % of a component of the ginkgo tree and 1-10 wt % dispersant (e. g. polyvinylpyrrolidone) are dissolved in up to 100 wt % organic solvent (e.g. cy-clohexanone). Dilution with water gives a dispersion.
iii) Emulsifiable concentrates (EC): 15-70 wt % of a component of the ginkgo tree and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.
iv) Emulsions (EW, EO, ES): 5-40 wt % of a component of the ginkgo tree and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.
v) Suspensions (SC, OD, FS): in an agitated ball mill, 20-60 wt % of a component of the ginkgo tree are comminuted with addition of 2-10 wt % dispersants and wetting agents (e.g. sodium lignosulfonate and alcohol ethoxylate), 0,1-2 wt % thickener (e.g. xanthan gum) and up to 100 wt % water to give a fine active substance suspension. Dilution with water gives a stable suspension of the active sub-stance. For FS type composition up to 40 wt % binder (e.g. polyvinylalcohol) is added.
vi) Water-dispersible granules and water-soluble granules (WG, SG): 50-80 wt % of a component of the ginkgo tree are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.
vii) Water-dispersible powders and water-soluble powders (WP, SP, WS): 50-80 wt % of a component of the ginkgo tree are ground in a rotor-stator mill with ad-dition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dis-persion or solution of the active substance.
viii) Gel (GW, GF): in an agitated ball mill, 5-25 wt % of a component of the ginkgo tree are comminuted with addition of 3-10 wt % dispersants (e.g. sodium lignosulfonate), 1-5 wt % thickener (e.g. carboxymethylcellulose) and up to 100 wt % water to give a fine suspension of the active sub-stance. Dilution with water gives a stable suspension of the active substance.
ix) Microemulsion (ME): 5-20 wt % of a component of the ginkgo tree are added to 5-30 wt % organic solvent blend (e.g. fatty acid dimethylamide and cyclohexanone), 10-25 wt % surfactant blend (e.g. alcohol ethoxylate and arylphenol ethoxylate), and water up to 100%. This mixture is stirred for 1 h to produce spontaneously a thermodynamically stable microemulsion.
x) Microcapsules (CS): an oil phase comprising 5-50 wt % of a component of the ginkgo tree, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a component of the ginkgo tree, 0-40 wt % water insoluble organic solvent (e.g. aromatic hydrocarbon), and an isocyanate monomer (e.g. diphenylmethene-4,4’-diisocyanatae) are dispersed into an aqueous solution of a protective colloid (e.g. polyvinyl alcohol). The addition of a polyamine (e.g. hexamethylenediamine) results in the formation of a polyurea microcapsule. The monomers amount to 1-10 wt %. The wt % relate to the total CS composition.
xi) Dustable powders (DP, DS): 1-10 wt % of a component of the ginkgo tree are ground finely and mixed intimately with up to 100 wt % solid carrier, e.g. finely divided kaolin.
xii) Granules (GR, FG): 0.5-30 wt % of a component of the ginkgo tree is ground finely and associated with up to 100 wt % solid carrier (e.g. silicate). Granulation is achieved by extrusion, spray-drying or the fluidized bed.
xiii) Ultra-low volume liquids (UL): 1-50 wt % of a component of the ginkgo tree are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising components of the ginkgo tree (or one of its individual embodiments) and/or future active substances may be mixed by the user in a spray tank and further auxiliaries and additives may be added, if appropriate.

In a further embodiment, either individual components of the composition according to the invention or partially premixed components can be applied jointly (e.g. after tank mix) or consecutively. The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures. The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “controlling” comprises both the combating of pests that have infested a cotton plant, and preventing the future infestation of the cotton plant. Preferably, controlling refers to the combating of pests that have infested a cotton plant.

The term “animal pest” includes arthropods, gastropods, and nematodes. Preferred animal pests are arthropods, preferably insects and arachnids, in particular insects. Insects, which are of particular relevance for crops, are typically referred to as crop insect pests.

The term “crop” refers to both, growing and harvested crops.

The term “plant propagation material” refers to all the generative parts of the plant such as seeds and vegetative plant material such as cuttings, which can be used for the multiplication of the plant. This includes seeds, roots, fruits, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, or in furrow application, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The components of the ginkgo tree are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with components of the ginkgo tree. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

In addition, the active compound may also be used for the treatment of seeds from plants, which have been modified by mutagenesis or genetic engineering, and which e.g. tolerate the action of herbicides or fungicides or insecticides. Such modified plants have been described in detail above.

Conventional seed treatment formulations include for example flowable concentrates (FS), solutions (LS), suspoemulsions (SE), powders for dry treatment (DS), water dispersible powders for slurry treatment (WS), water-soluble powders (SS), emulsion (ES), and (EC) and gel formulation (GF). These formulations can be applied to the seed diluted or undiluted. Application to the seeds is carried out before sowing, either directly on the seeds or after having pregerminated the latter. Preferably, the formulations are applied such that germination is not included. The active substance concentrations in ready-to-use formulations, which may be obtained after two-to-tenfold dilution, are preferably from 0.01 to 60% by weight, more preferably from 0.1 to 40% by weight.

In a preferred embodiment, a FS formulation is used for seed treatment. Typically, a FS formulation may comprise 1-800 g/l of active ingredient, 1-200 g/l surfactant, 0 to 200 g/l antifreezing agent, 0 to 400 g/l of binder, 0 to 200 g/l of a pigment and up to 1 litre of a solvent, preferably water.

Especially preferred FS formulations of the compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the components of the ginkgo tree are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising components of the ginkgo tree, or an agriculturally useful salt thereof, as defined herein. The amount of the components of the ginkgo tree or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

Advantages of the present invention are that the pesticidal activity of the components of the ginkgo tree may be synergistically enhanced by the insecticidal trait of a modified plant, in particular by insecticidal traits conveyed by genes of Bacillus thuringiensis. Furthermore, it has been found that the compounds of the present invention are suitable for preventing insects to become resistant to the insecticidal trait, or for combating pests, which already have become resistant to the insecticidal trait of a modified plant. Moreover, the compounds of the present invention are suitable for combating pests, against which the insecticidal trait is not effective, so that the complementary insecticidal activity of the components of the ginkgo tree can advantageously be used.

The following examples illustrate the invention.

### EXAMPLES

Bilobalide and ginkgolide A are commercially available (e.g. from Interchim).

Fibermax™ LibertyLink™ cotton corresponding to row A35 in Table B—is resistant to glufosinate by virtue of event LLCotton25.

Bollgard II™ cotton corresponding to row A39 in Table B has insect resistance by vertue of event MON15985.

Abbreviations: l is litre, ha is hectare, DAI is day(s) after infestation, ppm is parts per million.

Relative efficacy (E) values were calculated according to Abbot's formula

E=[(C−G)/C]×100, wherein

C is the number of alive insects in untreated controls, and G is the number of alive insects in plants treated with components of the ginkgo tree.

### Example-1

Cotton plants were grown to growth stage BBCH 12. The cotton plants were then sprayed with 300 l/ha of aqueous compositions comprising either no component of the ginkgo tree, 300 ppm of ginkgolide A, or 100 ppm of bilobalide. The plants were then infested with five insects (either adult, or 4^{th} instar nymphs) of Nezara viridula insects. Insects were contained on the plant using mesh organza bags. Three replicate measurement on different plants were made for each measurement. Three days after infestation, the number of live and dead insects was determined. The mean mortality values were gathered in Table D.

TABLE D
Mortality in percent of N viridula three davs after infestation

| | Mortality of Adults | Mortality of 4^{th} instar nymphs |
|---|---|---|
| Applied Compound | [%] | [%] |
| Untreated Control | 0 | 0 |
| Ginkgolide A | 93.3 | 78.6 |
| Bilobalide | 53.3 | 92.9 |

### Example-2

Fibermax™ LibertyLink™ cotton, and Bollgard II™ cotton seedlings were sprayed with 2 ml of compositions comprising either no component of the ginkgo tree, or bilobalide at a concentration of 150 ppm, or 100 ppm. Plants were allowed to dry and subsequently infested with ten adults of Euschistus heros. Four replicate measurement on different plants were made for each measurement. One day after infestation (1 DAI), and three days after infestation (3 DAI) the number of live and dead insects was determined. The mean number of alive insects, as well as the relative efficacy values (E) according to Abbott's formula were gathered in Table E, wherein the number of alive insects in untreated controls (C) was the value of the same plant without the application of bilobalide.

TABLE E
Control of E. heros in cotton by bilobalide

| | Concen- | No of | No of | Relative | Relative |
|---|---|---|---|---|---|
| | tration | alive | alive | Efficacy | Efficacy |
| | of Bilobalide | insects | insects | 1DAI | 3DAI |
| Cotton plant | [ppm] | 1DAI | 3DAI | [%] | [%] |
| Fibermax ™ | — | 10.0 | 10.0 | — | — |
| LibertyLink ™ | | | | | |
| Bollgard II ™ | — | 10.0 | 10.0 | — | — |
| Fibermax ™ | 150 | 3.0 | 0.0 | 70 | 100 |
| LibertyLink ™ | | | | | |
| Bollgard II ™ | 150 | 2.5 | 0.0 | 75 | 100 |
| Fibermax ™ | 100 | 3.3 | 0.3 | 68 | 98 |
| LibertyLink ™ | | | | | |
| Bollgard II ™ | 100 | 2.3 | 0.3 | 78 | 98 |

### Example-3

The experiment as described in Example-2 was repeated with ginkgolide A in a concentration of 250 ppm. The numbers of live and dead insects were determined three days after infestation (3DAI). The mean number of alive insects, as well as the relative efficacy values (E) according to Abbott's formula were gathered in Table F, wherein the number of alive insects in untreated controls (C) was the value of the same plant without the application of ginkgolide A.

TABLE F
Control of E. heros in cotton by ginkgolide A

| | Concen- | No of | No of | Relative | Relative |
|---|---|---|---|---|---|
| | tration | alive | alive | Efficacy | Efficacy |
| | of ginkgolide A | insects | insects | 1DAI | 3DAI |
| Cotton plant | [ppm] | 1DAI | 3DAI | [%] | [%] |
| Fibermax ™ | — | 10.0 | 10.0 | — | — |
| LibertyLink ™ | | | | | |
| Bollgard II ™ | — | 10.0 | 10.0 | — | — |
| Fibermax ™ | 250 | 10.0 | 9.5 | 0 | 5 |
| LibertyLink ™ | | | | | |
| Bollgard II ™ | 250 | 9.5 | 6.8 | 5 | 33 |

1. A method for controlling pests of a cotton plant, comprising the step of contacting the cotton plant, parts of it, or its propagation material with components of a ginkgo tree selected from bilobalide, ginkgolide A, ginkgolide B, ginkgolide C, ginkgolide J, ginkgolide M, and mixtures thereof.

2. The method according to claim 1, wherein the pests are from a family of Pentatomidae.

3. The method according to claim 1, wherein the pests are selected from the group consisting of Acrosternum spp., Euschistus spp., Halyomorpha spp., Nezara spp., Megacopta spp., and Piezodorus spp.

4. The method according to claim 1, wherein the pests are selected from the group consisting of Acrosternum hilare, Euschistus servus, Euschistus heros, Halyomorpha halys, Megacopta cribraria, Nezara viridula, and Piezodorus viridula.

5. The method according to claim 1, wherein the pests are Euschistus heros, or Nezara viridula.

6. The method according to claim 1, wherein the components of the ginkgo tree are applied by soil drench treatment.

7. The method according to claim 1, wherein the components of the ginkgo tree are applied by in-furrow treatment.

8. The method according to claim 1, wherein the components of the ginkgo tree are applied by foliar application.

9. The method according to claim 1, wherein the components of the ginkgo tree are applied in an amount of 1 to 500 g/ha.

10. The method according to claim 1, wherein the components of the ginkgo tree are selected from bilobalide, ginkgolide A, and mixtures thereof.

11. The method according to claim 1, wherein the cotton plant has been modified by mutagenesis or genetic engineering.

12. The method according to claim 1, wherein the cotton plant has an insect resistance conveyed by at least one gene selected from cry1A, cry1Ab, cry1Ac, cry1C, cry1F, cry2Ae, cry2Ab2, cry1Ab-Ac, vip3A(a), and CpTI, and combinations thereof.

13. The method according to claim 1, wherein the cotton plant has an insect resistance conveyed by at least one gene selected from cry1Ab, cry1Ac, cry1F, cry2Ab2, cry2Ae, vip3A(a), and combinations thereof.

14. The method according to claim 1 wherein the cotton plant has a herbicide tolerance conveyed by at least one gene selected from aad-12, bar, bxn, cp4 epsps, dmo, 2mepsps, pat, S4-hrA, and combinations thereof.

15. A pesticide comprising one or more components of a ginkgo tree selected from bilobalide, ginkgolide A, ginkgolide B, ginkgolide C, ginkgolide J and ginkgolide M for controlling pests on a cotton plant, parts of it, or its propagation material.
